(19) United States
(12) Patent Application Publication
(10) Pub. No.: US 2020/0289825 A1
(43) Pub. Date: Sep. 17, 2020

(54) Actuated Electrode Lead in Minimally Invasive Cochlear Implant Surgery

(71) Applicant: MED-EL Elektromedizinische Geraete GmbH, Innsbruck (AT)

(72) Inventors: Max Frölich, Hannover (DE); Daniel Schurzig, Hannover (DE)

(21) Appl. No.: 16/338,777

(22) PCT Filed: Nov. 1, 2017
(86) PCT No.: PCT/US2017/059433

§ 371 Date: Apr. 2, 2019

Related U.S. Application Data
(60) Provisional application No. 62/415,575, filed on Nov. 1, 2016.

(51) Int. Cl. A61N 1/36; A61N 1/05; A61N 1/372; A61N 1/375

(52) U.S. Cl. CPC A61N 1/36139; A61N 1/0541; A61N 1/3752; A61N 1/372; A61N 1/36038

(57) ABSTRACT

An implantable electrode arrangement for a cochlear implant system is described. An electrode lead contains a lead structure control element configured to control a lead shape of the electrode lead between an insertion state wherein the electrode lead within a mastoid tunnel has a longitudinal lead axis lying entirely along the tunnel axis, and a post-insertion state wherein a facial recess portion of the electrode lead fitting within a limited diameter portion of the mastoid tunnel associated with a facial recess portion of the mastoid bone has a longitudinal lead axis along the tunnel axis, and a storage portion of the electrode lead fitting within an enlarged diameter portion of the mastoid tunnel lateral to the facial recess portion has a longitudinal lead axis coiled in a helical shape radially around the tunnel axis.

This application claims priority from U.S. Provisional Patent 62/415,576, filed Nov. 1, 2016 and incorporated herein by reference in its entirety.

### TECHNICAL FIELD

The present invention relates to medical implants, and more specifically to an implantable electrode arrangement for cochlear implant systems.

### BACKGROUND ART

A normal ear transmits sounds as shown in FIG. 1 through the outer ear 101 to the tympanic membrane (eardrum) 102, which moves the bones of the middle ear 103, which in turn vibrate the oval window and round window openings of the cochlea 104. The cochlea 104 is a long narrow duct wound spirally about its axis for approximately two and a half turns. The cochlea 104 includes an upper channel known as the scala vestibuli and a lower channel known as the scala tympani, which are connected by the cochlear duct. The scala tympani forms an upright spiraling cone with a center called the modiolar where the spiral ganglion cells of the acoustic nerve 113 reside. In response to received sounds transmitted by the middle ear 103, the fluid filled cochlea 104 functions as a transducer to generate electric pulses that are transmitted to the cochlear nerve 113, and ultimately to the brain. Hearing is impaired when there are problems in the ability to transduce external sounds into meaningful action potentials along the neural substrate of the cochlea 104.

In some cases, hearing impairment can be addressed by an auditory prosthesis system such as a cochlear implant that electrically stimulates auditory nerve tissue with small currents delivered by multiple stimulation contacts distributed along an implant electrode. FIG. 1 shows some components of a typical cochlear implant system where an external microphone provides an audio signal input to an external signal processing stage 111 which implements one of various known signal processing schemes. The processed signal is converted by the external signal processing stage 111 into a digital data format, such as a sequence of data frames, for transmission into a receiver processor in an implant housing 108. Besides extracting the audio information, the receiver processor in the implant housing 108 may perform additional signal processing such as error correction, pulse formation, etc., and produces a stimulation pattern (based on the extracted audio information) that is sent through an electrode lead 109 to an implanted electrode array 110 which penetrates into the cochlea 104 through a surgical opening in the outer surface of the cochlea 104. Typically, this electrode array 110 includes multiple stimulation contacts 112 on its surface that deliver the stimulation signals to adjacent neural tissue of the cochlea 104 which the brain of the patient interprets as sound. The individual stimulation contacts 112 may be activated sequentially or simultaneously in one or more contact groups.

Cochlear implantation is a major surgery that involves full anesthesia and usually takes from 1.5 to 5 hours. A significant portion of that time is required for the labor intensive mastoidectomy in which the surgeon creates an opening in the outer mastoid bone of the skull and an electrode path through that bone and the middle ear to gain access to the cochlea prior to implantation. During this process, the surgeon needs to carefully mill down through the mastoid bone to the cochlea starting right behind the ipsilateral ear, and using anatomical landmarks to find his way. One of these landmarks is the facial nerve which, if damaged or cut, may cause facial paralysis of the patient. FIG. 2A shows an x-ray image of a cochlear implant electrode inserted into a patient cochlea via such a conventional mastoidectomy.

Aiming at the reduction of surgery time, patient stress, and risk of accidents such as facial nerve damage, there are research attempts to perform cochlear implantation using image guidance using preoperative CT images for the determination of a single bore path from behind the ear down to the point on the outer surface of the cochlea through which the implant electrode array needs to be inserted. These methods are described in detail, for example, in Labadie et al. “Minimally invasive, image-guided, facial-recess approach to the middle ear: demonstration of the concept of percutaneous cochlear access in vitro.” Otology & Neurotology 26.4 (2005): 557-562; which is incorporated herein by reference. FIG. 2B shows an x-ray image of a cochlear implant electrode inserted into a patient cochlea via this new minimally invasive technique. While these attempts are known to be very beneficial in terms of the severity of the surgery, the actual insertion of the electrode array into the cochlea becomes significantly more difficult—the geometrical boundary conditions do not allow for visual access of the cochlea opening, and there is little or no space available for surgical insertion mechanisms.

It is known that the length of the electrode lead does not correspond to the exact distance between the final location of the implant housing and the opening into the implanted cochlea. That is because the thickness of the mastoid bone varies between one patient and another. In addition, when using robotic surgery techniques to drill an electrode path to the cochlea, the drilled tunnels are so small that appropriate insertion tools are needed for safe insertion of the electrode array section into the cochlea. These insertion tools usually require some extra length of electrode lead beyond the minimum possible distance between the cochlea and the implant housing to provide appropriate gripping and handling options.

FIG. 3 shows the usual surgical technique for storing excess electrode lead in the mastoidectomy opening. The excess lead is looped into an 8- or O-shape in the cavity underneath the mastoid cortical overhang. There appears to be no existing discussion of how to store excess electrode lead length when using the newer minimally invasive bore path technique.

### SUMMARY

Embodiments of the present invention are directed to an implantable electrode arrangement for a cochlear implant system that is suitable for storage of excess electrode lead in a minimally invasive implantation surgery. An implantable electrode arrangement for a cochlear implant system is described. An electrode lead carries one or more cochlear stimulation signals and fits through a mastoid tunnel that extends along a longitudinal tunnel axis from a lateral outer surface of patient mastoid bone through the patient mastoid bone into the middle ear. An electrode array is connected to a distal end of the electrode lead and is inserted into a patient cochlea so that stimulation contacts on its outer surface can apply the cochlear stimulation signals to target neural tissue. A lead structure control element is located within the electrode lead to control a lead shape of the electrode lead between an insertion state wherein the electrode lead within the mastoid tunnel has a longitudinal lead axis lying entirely along the tunnel axis, and a post-insertion state wherein a facial recess portion of the electrode lead fitting within a limited diameter portion of the mastoid tunnel associated with a facial recess portion of the mastoid bone has a longitudinal lead axis along the tunnel axis, and a storage portion of the electrode lead fitting within an enlarged diameter portion of the mastoid tunnel lateral to the facial recess portion has a longitudinal lead axis coiled in a helical shape radially around the tunnel axis.

In further specific embodiments, the lead structure control element may include one or more shape memory alloy elements to control lead shape, and/or the lead structure control element may be configured to control lead shape as a function of temperature. For example, the lead structure control element may include one or more lead heating elements configured for heating the electrode lead.

Specific embodiments of the invention may further include a lead stopper connected to the electrode lead and configured to securely fit into the mastoid tunnel from the middle ear so as to resist rotation of electrode array when the lead structure control element controls the lead shape into the post-insertion state. For example, the lead stopper may be structurally integrated into the electrode lead, or it may be a structurally separate element securely attached to the electrode lead.

Embodiments of the present invention also include a method of implanting an electrode array in a patient cochlea. An outer mastoid tunnel is prepared that extends along a longitudinal tunnel axis from a lateral outer surface of a patient mastoid bone through the mastoid bone towards a facial recess region of the patient mastoid bone. An inner mastoid tunnel is prepared that extends further along the tunnel axis through the facial recess region of the mastoid bone into the middle ear. The inner mastoid tunnel diameter is less than the outer tunnel diameter. A cochlear opening also is prepared in an outer surface of a patient cochlea at a point along the tunnel axis. An implant electrode arrangement is provided that includes an electrode lead configured for carrying one or more cochlear stimulation signals and having a distal end connected to an electrode array with an outer surface having a plurality of stimulation contacts configured for applying the cochlear stimulation signals to target neural tissue within the patient cochlea. The electrode lead further comprises a lead structure control element configured to control lead shape. The electrode array is fitted through the outer mastoid tunnel, the inner mastoid tunnel, the middle ear, and the cochlear opening to implant the electrode array into the patient cochlea. The lead structure control element operates to maintain the electrode lead enclosed within the outer mastoid tunnel and the inner mastoid tunnel in an insertion shape lying entirely along a longitudinal lead axis extending along the tunnel axis. Then the lead structure control element is operated to modify lead shape of the electrode lead into a post-insertion shape wherein a facial recess portion of the electrode lead fitting within the inner mastoid tunnel has a longitudinal lead axis along the tunnel axis, and a storage portion of the electrode lead fitting within the outer mastoid tunnel has a longitudinal lead axis coiled in a helical shape radially around the tunnel axis.

In further specific embodiments, the lead structure control element may comprise one or more shape memory alloy elements to control lead shape, and/or the lead structure control element may be configured to control lead shape as a function of temperature. For example, the lead structure control element may include one or more lead heating elements configured for heating the electrode lead.

Specific embodiments may further include securely fitting a lead stopper connected to the electrode lead into the inner mastoid tunnel from the middle ear so as to resist rotation of electrode array when the lead structure control element operates to modify the lead shape into the post-insertion state. For example, the lead stopper may be structurally integrated into the electrode lead, or it may be a structurally separate element securely attached to the electrode lead.

Embodiments of the present invention also include a cochlear implant system having an electrode arrangement according to any of the foregoing.

### BRIEF DESCRIPTION OF THE DRAWINGS

FIG. 1 shows various anatomical structures in a human ear and some components of a typical cochlear implant system.

FIGS. 2A and 2B show examples of x-ray images for implanting a cochlear implant electrode using a conventional mastoidectomy and a minimally invasive mastoid tunnel respectively.

FIG. 3 shows conventional storage of excess electrode lead in a mastoidectomy opening.

FIG. 4 shows anatomical details of a mastoid tunnel suitable for excess electrode storage according to an embodiment of the present invention.

FIG. 5 shows various logical steps in a method of surgically inserting a cochlear implant electrode array according to an embodiment of the present invention.

FIGS. 6A-6B show structural details of the surgical insertion process for a cochlear implant electrode according to an embodiment of the present invention.

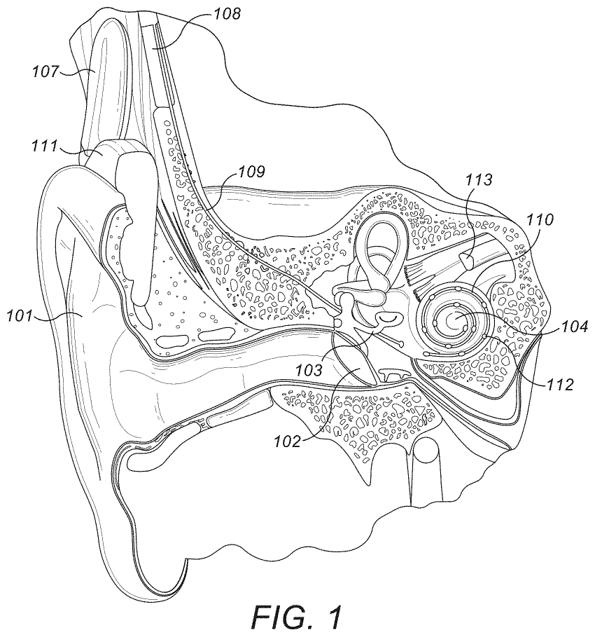

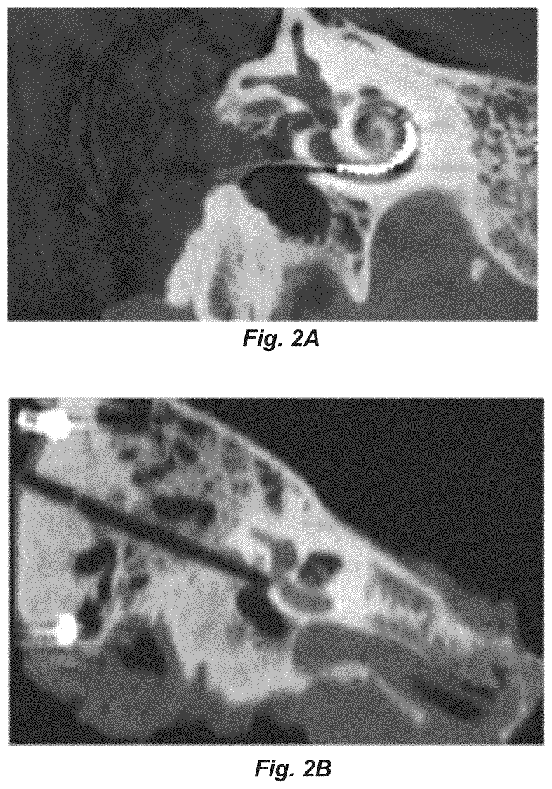

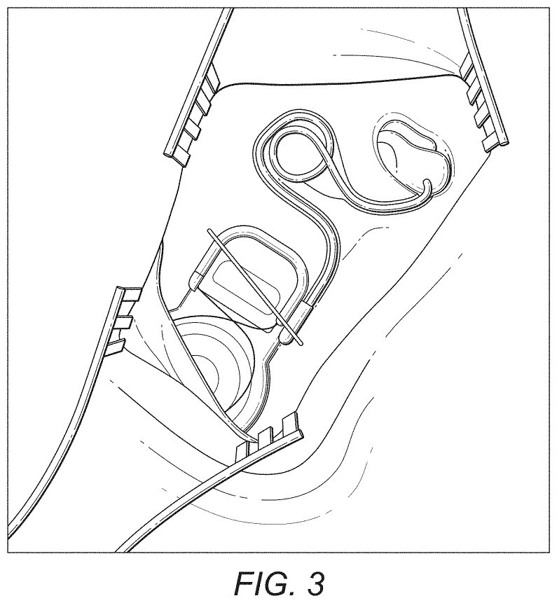

### DETAILED DESCRIPTION

Embodiments of the present invention are directed to a cochlear implant electrode lead suitable for implementing a new storage technique for excess electrode lead consistent with a minimally invasive implantation approach without needing to create an additional storage cavity.

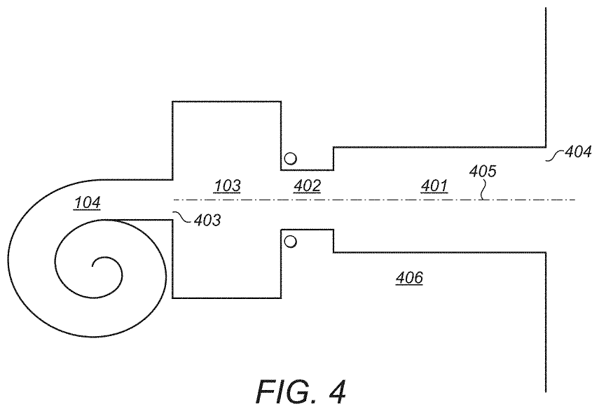

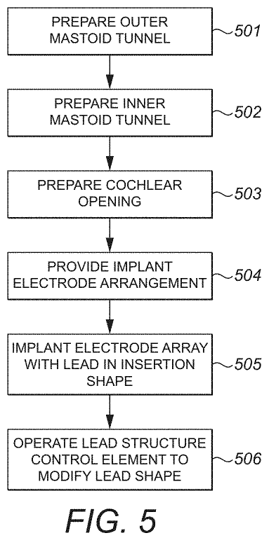

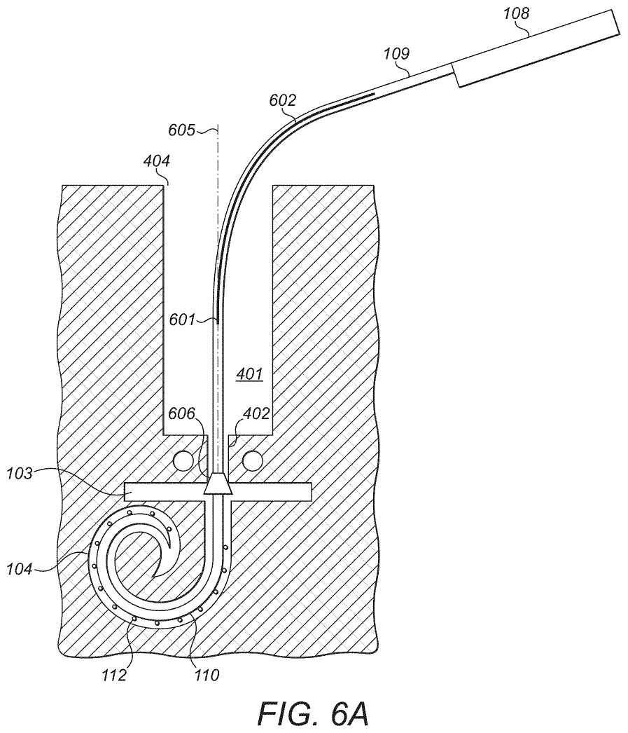

FIG. 4 shows anatomical details of a mastoid tunnel 400 suitable for excess electrode storage and FIG. 5 shows various logical steps in a method of surgically inserting a cochlear implant electrode array according to an embodiment of the present invention. The mastoid tunnel 400 extends along a longitudinal tunnel axis 405 from a lateral outer surface of patient mastoid bone 404 through the patient mastoid bone 406 into the middle ear 103. Initially an enlarged diameter outer mastoid tunnel 401 (e.g., about 3.0 mm in diameter) is prepared, step 501, that is drilled during implantation surgery through the mastoid bone 406 to near the critical facial recess structures of the facial nerve and the chorda tympani, terminating the tunnel before these structures are traumatized or harmed (e.g., about 24.0 mm long). After checking the drilled path, a narrower inner mastoid tunnel 402 (e.g., about 1.8 mm in diameter) then is drilled through the facial recess region of the mastoid bone 406 into the middle ear 103, step 502. A cochlear opening 403 is then created in the outer surface of the patient cochlea 104 at a point on the longitudinal tunnel axis 405, step 503.

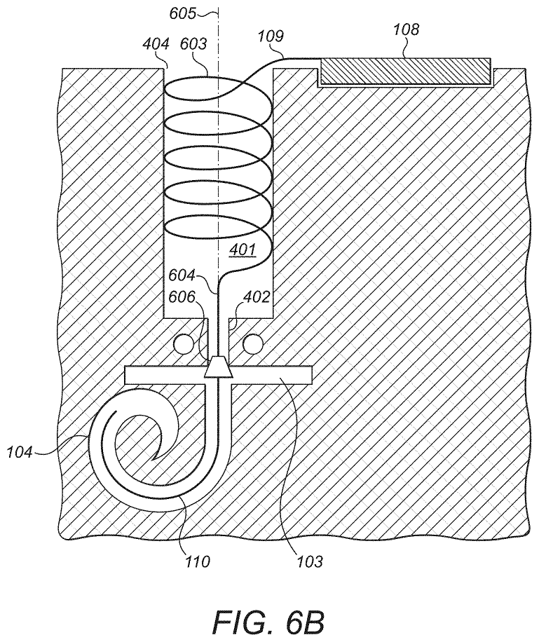

FIGS. 6A-6B show structural details of the surgical insertion process for a cochlear implant electrode according to an embodiment of the present invention. An implant electrode is provided, step 504, that includes an electrode lead 109 for carrying one or more cochlear stimulation signals. An electrode array 110 is connected to a distal end of the electrode lead 109 and includes stimulation contacts 112 on its outer surface configure to apply the cochlear stimulation signals to target neural tissue within the implanted cochlea 104. A lead structure control element 602 also is located within the electrode lead 109 to control the lead shape of the electrode lead 109. The electrode lead 109 and electrode array 110 are held in an insertion tool (not shown) with the lead control element 602 is operated to maintain an insertion lead shape as shown in FIG. 6A wherein the electrode lead 109 within the outer mastoid tunnel 401 and the inner mastoid tunnel 402 has a longitudinal lead axis lying entirely along the tunnel axis 605. Once the electrode array 110 is fully implanted in the cochlea 104, step 505, the insertion tool is removed the implant housing 108 can be secured in its final position. The lead control element 602 is then operated to modify the lead shape into a post-insertion state, step 506, in which a facial recess portion 604 of the electrode lead 109 within the limited diameter portion of the inner mastoid tunnel 402 continues to have its longitudinal lead axis along the tunnel axis 605, while a storage portion 603 of the electrode lead 109 fitting within the enlarged diameter outer mastoid tunnel 401 (lateral to the facial recess portion inner mastoid tunnel 402) has its longitudinal lead axis coiled in a helical shape radially around the tunnel axis 605.

The lead control element 602 specifically may be composed of one or more shape memory alloy (SMA) elements; for example, materials such as nickel titanium alloys. The SMA elements of the lead control element 602 can be modified out of their “memorized” shape at a first lower temperature. Then the SMA elements can be heated to modify them back into their predefined shape. For example, the heating and the specific transformation temperature of the SMA elements may be based on body temperature. In that case, the SMA elements are passively heated by virtue of their proximity to the surrounding body tissue. Alternatively, the transformation temperature for the SMA elements may lie above body temperature, though preferably only slightly above body temperature. A controlled current then can be applied by one or more dedicated lead heating elements embedded in the electrode lead, where the time and speed of shape transformation can be controlled by the surgeon. The power supply for the control current could be provided via the implant processor or externally. For an external power supply, the extra current supply wire would have to be cut before suturing and closing the implantation wound. Increasing the lead temperature above body temperature during the surgery is acceptable because the heating location is sufficiently far away from the delicate inner ear tissues, which are known to be very sensitive to damage from elevated temperatures. And also the structures that are heated are embedded in the silicone material of the electrode lead, which acts as a heat insulator with a relatively low thermal expansion coefficient.

For the post-insertion state of the stored portion of the electrode lead, the helix shape will automatically start to coil within a defined coiling radius R_{L} like in an old-fashioned telephone handset (e.g., 1.25 mm≤R_{L}≥5 mm), which is slightly smaller than the diameter of the outer mastoid tunnel (2R_{L}<T_{D}). The resulting coiling action reduces the length of the electrode lead so that the excess length self-stores into the drilled path of the outer mastoid tunnel, and the implant body can be placed close to the drilled tunnel with no additional drilling of a grove needed.

To control the coiling length and adapt the position of the implant body, the shape structure control element can be divided into multiple different SMA segments, each of which may be controlled separately and which may allow for different coiling radii. In addition or alternatively, the SMA segments can be manually deformed into any desired form and the actuation and shape modification process can be repeated if needed.

It will be appreciated that during the active coiling of the electrode lead, a torque is created that tends to rotate the more distal structures (i.e. the intracochlear electrode array), and/or the more proximal structures (i.e. the implant housing). Rotation of the distal structures needs to be absolutely avoided since rotation of the electrode array within the cochlea would seriously traumatize the delicate tissue therein. To prevent that, embodiments of the invention may include a lead stopper (605 in FIGS. 6A-6B) that is connected to the electrode lead and configured to securely fit into the mastoid tunnel from the middle ear so as to resist rotation of electrode array when the lead structure control element controls the lead shape into the post-insertion state. For example, the lead stopper 605 may be structurally integrated into the electrode lead, or it may be a structurally separate element securely attached to the electrode lead. Once the lead stopper 605 is secured in the tunnel opening, coiling of the a storage portion 603 of the electrode lead 109 within the enlarged diameter outer mastoid tunnel 401 creates a rotational force on the entire more proximal portion of the electrode lead 109 and the implant housing 108. As long as the coiling rate is controlled, the surgeon and/or an appropriate tool can manage this rotational force until the coiling procedure is completed.

The mechanical load onto the electrode lead caused by manipulation by hand and/or surgical tools will be reduced compared to the manipulation in conventional surgical techniques. This is mainly due to the automatic coiling, and thus no additional manual handling is required. And if the coiling radius of the storage portion 603 of the electrode lead 109 within the enlarged diameter outer mastoid tunnel 401 is larger than a critical bending radius for the wires embedded in the electrode lead, there should be no need to consider or account for any breakage of the lead wires.

Although various exemplary embodiments of the invention have been disclosed, it should be apparent to those skilled in the art that various changes and modifications can be made which will achieve some of the advantages of the invention without departing from the true scope of the invention.

1. An implantable electrode arrangement for a cochlear implant system comprising:
an electrode lead configured for carrying one or more cochlear stimulation signals and configured to fit through a mastoid tunnel extending along a longitudinal tunnel axis from a lateral outer surface of patient mastoid bone through the patient mastoid bone into the middle ear;
an electrode array connected to a distal end of the electrode lead and configured for insertion into a patient cochlea, wherein the electrode array has an outer surface with a plurality of stimulation contacts configured for applying the cochlear stimulation signals to target neural tissue within the patient cochlea; and
a lead structure control element within the electrode lead configured to control a lead shape of the electrode lead between two stable states:
an insertion state wherein the electrode lead within the mastoid tunnel has a longitudinal lead axis lying entirely along the tunnel axis; and
a post-insertion state wherein:
a facial recess portion of the electrode lead fitting within a limited diameter portion of the mastoid tunnel associated with a facial recess portion of the mastoid bone has a longitudinal lead axis along the tunnel axis, and
a storage portion of the electrode lead fitting within an enlarged diameter portion of the mastoid tunnel lateral to the facial recess portion has a longitudinal lead axis coiled in a helical shape radially around the tunnel axis.

2. The electrode arrangement according to claim 1, wherein the lead structure control element comprises one or more shape memory alloy elements to control lead shape.

3. The electrode arrangement according to claim 1, wherein the lead structure control element is configured to control lead shape as a function of temperature.

4. The electrode arrangement according to claim 3, wherein the lead structure control element includes one or more lead heating elements configured for heating the electrode lead.

5. The electrode arrangement according to claim 1, further comprising:
a lead stopper connected to the electrode lead and configured to securely fit into the mastoid tunnel from the middle ear so as to resist rotation of electrode array when the lead structure control element controls the lead shape into the post-insertion state.

6. The electrode arrangement according to claim 5, wherein the lead stopper is structurally integrated into the electrode lead.

7. The electrode arrangement according to claim 5, wherein the lead stopper is a structurally separate element securely attached to the electrode lead.

8. A cochlear implant system having an electrode arrangement according to any of claims 1-7.

9. A method of implanting a cochlear implant electrode in a patient, the method comprising:
preparing an outer mastoid tunnel extending along a longitudinal tunnel axis from a lateral outer surface of a patient mastoid bone through the mastoid bone towards a facial recess region of the patient mastoid bone, wherein the outer mastoid tunnel is characterized by an outer tunnel diameter;
preparing an inner mastoid tunnel along the tunnel axis through the facial recess region of the mastoid bone into the middle ear, wherein the inner mastoid tunnel is characterized by an inner tunnel diameter less than the outer tunnel diameter;
preparing a cochlear opening in an outer surface of a patient cochlea at a point along the tunnel axis;
providing an implant electrode arrangement comprising an electrode lead configured for carrying one or more cochlear stimulation signals and having a distal end connected to an electrode array with an outer surface having a plurality of stimulation contacts configured for applying the cochlear stimulation signals to target neural tissue within the patient cochlea, wherein the electrode lead further comprises a lead structure control element configured to control lead shape;
fitting the electrode array through the outer mastoid tunnel, the inner mastoid tunnel, the middle ear, and the cochlear opening to implant the electrode array into the patient cochlea, wherein the lead structure control element operates to maintain the electrode lead enclosed within the outer mastoid tunnel and the inner mastoid tunnel in an insertion shape lying entirely along a longitudinal lead axis extending along the tunnel axis; and
operating the lead structure control element to modify lead shape of the electrode lead into a post-insertion shape wherein:
a facial recess portion of the electrode lead fitting within the inner mastoid tunnel has a longitudinal lead axis along the tunnel axis, and
a storage portion of the electrode lead fitting within the outer mastoid tunnel has a longitudinal lead axis coiled in a helical shape radially around the tunnel axis.

10. The method according to claim 9, wherein the lead structure control element comprises one or more shape memory alloy elements to control lead shape.

11. The method according to claim 9, wherein the lead structure control element is configured to control lead shape as a function of temperature.

12. The method according to claim 11, wherein the lead structure control element includes one or more lead heating elements configured for heating the electrode lead.

13. The method according to claim 9, further comprising:
securely fitting a lead stopper connected to the electrode lead into the inner mastoid tunnel from the middle ear so as to resist rotation of electrode array when the lead structure control element operates to modify the lead shape into the post-insertion state.

14. The method according to claim 13, wherein the lead stopper is structurally integrated into the electrode lead.

15. The method according to claim 13, wherein the lead stopper is a structurally separate element securely attached to the electrode lead.
